(19) United States
(12) Patent Application Publication
(10) Pub. No.: US 2026/0043786 A1
(43) Pub. Date: Feb. 12, 2026

(54) ADJUSTMENT METHOD, ADJUSTMENT DEVICE, METHOD FOR MANUFACTURING MEASUREMENT DEVICE, AND MEASUREMENT DEVICE

(71) Applicant: YAMASHIN-FILTER CORP., Kanagawa (JP)

(72) Inventors: Nobuki SASAKI, Kanagawa (JP); Nobuyuki KITAJIMA, Kanagawa (JP)

(21) Appl. No.: 19/325,640

(22) Filed: Sep. 11, 2025

Related U.S. Application Data
(63) Continuation of application No. PCT/JP2024/008866, filed on Mar. 7, 2024.

(30) Foreign Application Priority Data
Mar. 15, 2023 (JP) 2023-040487

(51) Int. Cl. G01N 33/28; G01N 21/59; G01N 33/18

(52) U.S. Cl. CPC G01N 33/28; G01N 21/59; G01N 33/18

(57) ABSTRACT

A measurement device includes a light irradiation unit configured to irradiate a measurement flow path with light, and a light receiving unit configured to continuously receive the light emitted from the light irradiation unit and having passed through the measurement flow path, and to obtain a continuous electric signal. The measurement device is configured to measure a contamination level of oil based on an electric signal obtained by the light receiving unit in a state in which the oil flows through the measurement flow path. The light irradiation unit emits light in a state in which no oil flows through the measurement flow path, using a first pseudo signal for emitting light equivalent to light input to the light receiving unit when oil having a first contamination level as a given contamination level flows through the measurement flow path.

### CROSS-REFERENCE TO RELATED APPLICATIONS

This application is a continuation application of International Patent Application No. PCT/JP2024/008866 filed on Mar. 7, 2024, which claims priority to Japanese Patent Application No. 2023-040487 filed on Mar. 15, 2023, the entire contents of which are incorporated by reference.

### TECHNICAL FIELD

The present invention relates to an adjustment method, an adjustment device, a method for manufacturing a measurement device, and a measurement device.

### BACKGROUND ART

Patent Document 1 discloses a measurement device including a light irradiation unit that continuously irradiate liquid with light, a light receiving unit that continuously receives the light continuously emitted from the light irradiation unit and having passed through the liquid and converts the continuously received light into a continuous electric signal, a particle detection unit that amplifies the continuous electric signal converted by the light receiving unit at a first magnification to generate a continuous signal as a particle detection signal, an air bubble detection unit that amplifies the continuous electric signal converted by the light receiving unit at a second magnification smaller than the first magnification to generate a continuous signal as an air bubble detection signal, and a contamination level measurement unit that generates a signal for measuring a contamination of a liquid based on the particle detection signal and the air bubble detection signal.

### CITATION LIST

### Patent Literature

Patent Document 1: JP 6367649 B

In the measurement device described in Patent Document 1, the light receiving unit needs to be adjusted before measuring the contamination level. The adjustment of the light receiving unit is performed based on an electric signal obtained by continuously emitting light from the light irradiation unit in a state in which a liquid (oil such as hydraulic oil) having a known contamination level flows through the measurement flow path in advance and receiving the light by the light receiving unit. However, flowing the oil through measurement flow path requires time and effort to clean the oil and make adjustments.

### SUMMARY OF INVENTION

One or more aspects of the present invention provide an adjustment method in which a measurement device can be adjusted without using oil, an adjustment device, a method for manufacturing a measurement device, and a measurement device.

According to one or more aspects of the present invention, an adjustment method for adjusting a measurement device includes, for example, a light irradiation unit configured to irradiate a measurement flow path with light, and a light receiving unit configured to continuously receive the light emitted from the light irradiation unit and having passed through the measurement flow path, and to obtain a continuous electric signal. The measurement device is configured to measure a contamination level of oil based on an electric signal obtained by the light receiving unit in a state in which the oil flows through the measurement flow path, the adjustment method including a first adjustment step of emitting light from the light irradiation unit in a state in which no oil flows through the measurement flow path, using a first pseudo signal for emitting light equivalent to light input to the light receiving unit when oil having a first contamination level as a given contamination level flows through the measurement flow path, and obtaining a measurement value for the first contamination level based on an electric signal obtained by receiving the emitted light by the light receiving unit.

An adjustment device according to another aspect of the present invention adjusts a measurement device including, for example, a light irradiation unit configured to irradiate a measurement flow path with light, and a light receiving unit configured to continuously receive the light emitted from the light irradiation unit and having passed through the measurement flow path, and to obtain a continuous electric signal, the measurement device being configured to measure a contamination level of oil based on an electric signal obtained by the light receiving unit in a state in which the oil flows through the measurement flow path, the adjustment device including: an irradiation control unit configured to cause the light irradiation unit to emit light in a state in which no oil flows through the measurement flow path, using a first pseudo signal for emitting light equivalent to light input to the light receiving unit when oil having a first contamination level as a given contamination level flows through the measurement flow path; and a measurement unit configured to obtain a measurement value for the first contamination level based on an electric signal obtained by receiving the light emitted from the light irradiation unit by the light receiving unit.

A method for manufacturing a measurement device according to another aspect of the present invention is a method for manufacturing a measurement device configured to measure, in a state in which oil flows through a measurement flow path including a pipe, a contamination level of the oil based on an electric signal obtained by continuously receiving light, by a light receiving unit, emitted from a light irradiation unit configured to irradiate the measurement flow path with light, and having passed through the measurement flow path, the method including the steps of: providing the light irradiation unit and the light receiving unit to sandwich the pipe; causing the light irradiation unit to emit light in a state in which no oil flows through the measurement flow path, using a first pseudo signal for emitting light equivalent to light input to the light receiving unit when oil having a first contamination level as a given contamination level flows through the measurement flow path, and obtaining a measurement value for the first contamination level based on an electric signal obtained by receiving the emitted light by the light receiving unit; and assembling a housing in which the measurement flow path, the light irradiation unit, and the light receiving unit are provided.

In any of the above aspects of the present invention, the measurement device is adjusted by obtaining the measurement value for the first contamination level based on the electric signal obtained by receiving the light emitted from the light irradiation unit by the light receiving unit in the state in which no oil flows through the measurement flow path, using the first pseudo signal for emitting light equivalent to the light input to the light receiving unit when the oil having the given contamination level (first contamination level) flows through the measurement flow path. This allows for the adjustment of the measurement device without using oil.

The adjustment method may further include a pseudo signal generating step of generating the first pseudo signal using a reference measurement device, in which the reference measurement device includes a reference light irradiation unit configured to continuously irradiate a reference measurement flow path with light, and a reference light receiving unit configured to continuously receive the light continuously emitted from the reference light irradiation unit and having passed through the measurement flow path, and to obtain a continuous electric signal, the pseudo signal generating step may include a first reference value measurement step of emitting light from the reference light irradiation unit in a state in which oil having the first contamination level flows through the reference measurement flow path, measuring an electric signal obtained by receiving the emitted light by the reference light receiving unit, and obtaining a first reference measurement value for the first contamination level, and a first pseudo signal obtaining step of emitting light from the reference light irradiation unit, using a blinking signal for causing the reference light receiving unit to blink in a state in which no oil flows through the measurement flow path, and setting the blinking signal as the first pseudo signal when the electric signal obtained by the reference light receiving unit is equivalent to the electric signal having been obtained by the reference light receiving unit. This improves the accuracy of the first pseudo signal.

The first reference value measurement step may include obtaining the first reference measurement value by smoothing an integrated value per unit time of the electric signal obtained by receiving light by the reference light receiving unit, and the first pseudo signal obtaining step may include setting the blinking signal as the first pseudo signal when the integrated value per unit time of the electric signal obtained by receiving light by the reference light receiving unit is equivalent to the first reference measurement value. This allows the accurate first pseudo signals to be obtained and improves the adjustment accuracy.

The first pseudo signal obtaining step may include, in a state in which no oil flow through the measurement flow path, adjusting an output level of the reference light irradiation unit in such a manner that a maximum value of an output value of a signal obtained by the reference light receiving unit is equivalent to a reference output value that is a maximum value of the electric signal obtained by receiving light by the reference light receiving unit in the first reference value measurement step, and after the adjusting of the output level, emitting light from the reference light irradiation unit and obtaining the first pseudo signal. This allows the accurate first pseudo signal to be obtained and improves the adjustment accuracy.

The first adjustment step may include adjusting the output level of the light irradiation unit based on an adjustment result of the output level in the first pseudo signal obtaining step, and emitting light from the light irradiation unit, using the first pseudo signal at the output level having been adjusted. This improves the adjustment accuracy.

The measurement flow path may include a pipe at least partly made of glass, and the state in which no oil flows through the measurement flow path may be a state in which no liquid flows through the measurement flow path, a state in which a liquid other than oil flows through the measurement flow path, or a state in which a glass rod is provided instead of the pipe. The state in which a liquid other than oil flows through the measurement flow path may be a state in which water, alcohol, or a mixture of water and alcohol flows through the measurement flow path. In the state in which no liquid flows through the measurement flow path, the time for adjustment can be particularly shortened. The state in which a liquid other than oil flows through the measurement flow path or the state in which the glass rod is provided instead of the pipe is close to a state in which the oil flows through the pipe, and thus, the adjustment accuracy can be improved more easily while the time for adjustment is shortened.

To solve the above problem, an adjustment method according to the present invention is an adjustment method for adjusting a measurement device including, for example, a light irradiation unit configured to irradiate a measurement flow path with light, and a light receiving unit configured to continuously receive the light emitted from the light irradiation unit and having passed through the measurement flow path, and to obtain a continuous electric signal, the measurement device being configured to measure a contamination level of oil in a state in which the oil flows through the measurement flow path based on an electric signal obtained by the light receiving unit, the adjustment method including a first measurement step of continuously emitting light from the light irradiation unit, while moving a rod-shaped member, provided with a plurality of light-blocking slits that do not transmit light, at a first speed within the measurement flow path in such a manner that the light is equivalent to light input to the light receiving unit when oil having a first contamination level as a given contamination level flows through the measurement flow path, and obtaining a measurement value for the first contamination level based on an electric signal obtained by receiving the light by the light receiving unit upon the emission.

An adjustment device according to another aspect of the present invention is an adjustment device that adjusts a measurement device including, for example, a light irradiation unit configured to irradiate a measurement flow path with light, and a light receiving unit configured to continuously receive the light emitted from the light irradiation unit and having passed through the measurement flow path, and to obtain a continuous electric signal, the measurement device being configured to measure a contamination level of oil based on an electric signal obtained by the light receiving unit in a state in which oil flows through the measurement flow path, the adjustment device including: an irradiation control unit configured to cause the light irradiation unit to continuously emit light, while moving a rod-shaped member, provided with a plurality of light-blocking slits that do not transmit light, at a first speed within the measurement flow path in such a manner that the light is equivalent to light input to the light receiving unit when oil having a first contamination level as a given contamination level flows through the measurement flow path; and a measurement unit configured to obtain a measurement value for the first contamination level based on an electric signal obtained by receiving the light emitted from the light irradiation unit by the light receiving unit.

A method for manufacturing a measurement device according to another aspect of the present invention is a method for manufacturing a measurement device configured to measure, in a state in which oil flows through a measurement flow path including a pipe, a contamination level of oil based on an electric signal obtained by continuously receiving light, by a light receiving unit, emitted from a light irradiation unit configured to irradiate the measurement flow path with light, and having passed through the measurement flow path, the method including the steps of: providing the light irradiation unit and the light receiving unit to sandwich the pipe; causing the light irradiation unit to continuously emit light in a state in which no oil flows through the measurement flow path, while moving a rod-shaped member, provided with a plurality of light-blocking slits that do not transmit light, at a first speed within the measurement flow path in such a manner that the light is equivalent to light input to the light receiving unit when oil having a first contamination level as a given contamination level flows through the measurement flow path, and obtaining a measurement value for the first contamination level based on an electric signal obtained by receiving the light by the light receiving unit upon the emission; and assembling a housing in which the measurement flow path, the light irradiation unit, and the light receiving unit are provided.

In any of the above aspects of the present invention, the measurement device is adjusted by continuously emitting the light from the light irradiation unit, while moving the rod-shaped member, provided with the plurality of light-blocking slits that do not transmit light, within the measurement flow path at a first speed in such a manner that the light received by the light receiving unit is equivalent to the light input to the light receiving unit when the oil having the given contamination level (first contamination level) flows through the measurement flow path, and by obtaining the measurement value for the first contamination level based on the electric signal obtained by receiving the light by the light receiving unit upon the emission. This allows for the adjustment of the measurement device without using oil.

The adjustment method may further include a rod-shaped member adjustment step of adjusting a size and the number of the light-blocking slits and the first speed of the rod-shaped member, using a reference measurement device, in which the reference measurement device includes a reference light irradiation unit configured to continuously irradiate a reference measurement flow path with light, and a reference light receiving unit configured to continuously receive light continuously emitted from the reference light irradiation unit and having passed through the measurement flow path, and to obtain a continuous electric signal, the rod-shaped member adjustment step includes a first reference value measurement step of emitting light from the reference light irradiation unit in a state in which oil having the first contamination level flows through the reference measurement flow path, measuring an electric signal obtained by receiving the emitted light by the reference light receiving unit, and obtaining a first reference measurement value for the first contamination level, and an adjustment step of continuously turning on light from the reference light irradiation unit, obtaining an electric signal by the reference light receiving unit, while moving a provisional rod-shaped member, provided with a given size and number of the light-blocking slits, at a given provisional speed within the reference measurement flow path, and setting the provisional rod-shaped member and the provisional speed as the rod-shaped member and the first speed, respectively, when the electric signal is equivalent to the first reference measurement value. This improves the adjustment accuracy of the rod-shaped member and the first speed.

The first reference value measurement step may include obtaining the first reference measurement value by smoothing an integrated value per unit time of the electric signal obtained by receiving light by the reference light receiving unit, and the adjustment step may include setting the provisional rod-shaped member and the provisional speed as the rod-shaped member and the first speed, respectively, when the integrated value per unit time of the electric signal obtained by receiving light by the reference light receiving unit is equivalent to the first reference measurement value. This improves the adjustment accuracy.

According to one or more aspects of the present invention, the measurement device can be adjusted without using oil.

### BRIEF DESCRIPTION OF DRAWINGS

FIG. 1 is a cross-sectional view schematically illustrating a measurement device 1.

FIG. 2 is a block diagram schematically illustrating an electrical configuration of the measurement device 1, 1A, and an adjustment device 2 connected to the measurement device 1,1A.

FIG. 3 is a flowchart illustrating a process flow in which the adjustment device 2 performs adjustment processing of the measurement device 1A.

FIG. 4 is a flowchart illustrating a process flow of a method for manufacturing the measurement device 1A.

FIG. 5 is a schematic diagram for explaining a method for obtaining a measurement value, in which (A) schematically illustrates a flow of particles (dust) contained in oil flowing through a pipe 39, and (B) schematically illustrates a temporal change in an output of an electric signal.

FIG. 6 is a flowchart illustrating a process flow for obtaining a pseudo signal (step SP20).

FIG. 7 illustrates a state of light emitted from a light emitting element 11, in which (A) illustrates a state in which oil is present in the pipe 39, and (B) illustrates a state in which no oil is present and air is present in the pipe 39.

FIG. 8 schematically illustrates an electric signal obtained by a light receiving unit 20 when the light emitting element 11 blinks using a blinking signal sn.

FIG. 9 is a flowchart illustrating a process flow of step SP30.

FIG. 10 schematically illustrates a state in which the measurement device 1 obtains a pseudo signal and the measurement device 1A obtains a measurement value.

FIG. 11 is a block diagram schematically illustrating an electrical configuration of the measurement device 1, 1A and an adjustment device 2A connected to the measurement device 1, 1A.

FIG. 12 is a flowchart illustrating a process flow in which the adjustment device 2A performs adjustment processing of the measurement device 1A.

FIG. 13 is a flowchart illustrating a process flow for a rod-shaped member adjustment step (step SP40).

FIG. 14 is a flowchart illustrating a process flow of step SP50.

FIG. 15 is a block diagram schematically illustrating an electrical configuration of a measurement device 1B and an adjustment device 2 connected to the measurement device 1B.

FIG. 16 is a flowchart illustrating a process flow in which the adjustment device 2 performs adjustment processing of the measurement device 1B.

### DESCRIPTION OF EMBODIMENTS

Embodiments of the present invention will be described below in detail with reference to the drawings. The present invention relates to adjustment of a measurement device installed at a desired location of a device that performs desired operations using oil, such as construction machinery, hydraulic equipment, and the like, to measure a contamination level of the oil. The adjustment of the measurement device is performed in a manufacturing process before shipment of the measurement device.

### First Embodiment

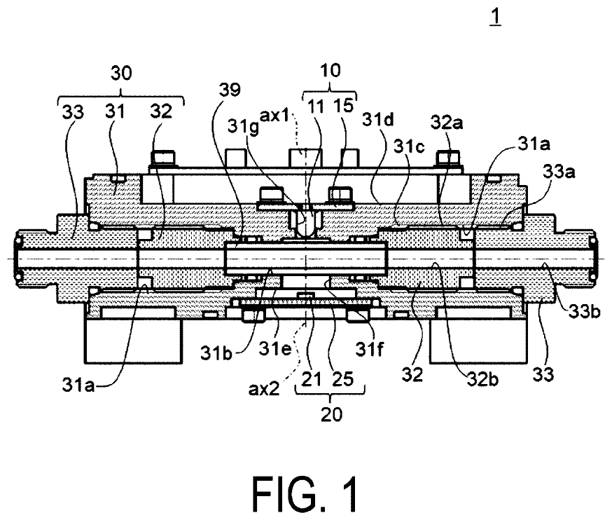

FIG. 1 is a cross-sectional view schematically illustrating a measurement device 1. In FIG. 1, hatching illustrating cross sections is partly omitted. The measurement device 1 mainly includes a light irradiation unit 10, a light receiving unit 20, a housing 30, and a pipe 39.

The light irradiation unit 10 mainly includes a light emitting element 11 and a substrate 15 on which the light emitting element 11 is provided. The light emitting element 11 is, for example, an LED that emits light toward the pipe 39.

The light receiving unit 20 mainly includes a light receiving element 21 and a substrate 25 on which the light receiving element 21 is provided. The light receiving element 21 is, for example, a photo diode (PD) that detects transmitted light by irradiation of light.

The light emitting element 11 and the light receiving element 21 are disposed to sandwich the pipe 39. An optical axis ax1 of the light emitting element 11 overlaps a light receiving region of the light receiving element 21. The light receiving region is a region where incident light can be detected, and the light receiving element 21 converts the light incident on the light receiving region into an electric signal. For example, when the light receiving element 21 is a photo diode, the light receiving region is an inner region surrounded by an annular electrode.

In FIG. 1, the optical axis ax1 of the light emitting element 11 and the optical axis ax2 of the light receiving element 21 match, but the optical axis ax1 and the optical axis ax2 may not match.

The pipe 39 is at least partly made of a light transmissive material (here, glass), and a liquid such as oil or water to be measured passes therethrough. Part of the pipe 39 made of a light-transmissive material is irradiated with light from the light emitting element 11 on one side, while the light is received by the light receiving element 21 on the opposite side.

The pipe 39 may be entirely made of a light transmissive material, or may partly include a window for guiding light in and out. In FIG. 1, the pipe 39 is a glass tube entirely made of glass.

The pipe 39 is provided inside a housing 30. The housing 30 mainly includes a first housing 31, a second housing 32, and a third housing 33.

The first housing 31 includes bores 31a formed at both ends and a hole 31b communicating with the two bores 31a. The central axes of the bores 31a and the central axis of the hole 31b substantially match.

The pipe 39 is inserted into the hole 31b, and the second housing 32 is inserted into each of the bores 31a. The third housing 33 is partly inserted into the bores 31a on the outside of the second housing 32. A female thread portion 31c is formed in the bores 31a, into which male thread portions 32a, 33a formed on the outer peripheral surfaces of the second housing 32 and the third housing 33, respectively, to dispose the second housing 32 and the third housing 33 in the bores 31a. Thus, the housing 30 is assembled.

The second housing 32 and the third housing 33 include a hole 32b and a hole 33b, respectively. The holes 32b, 33b communicate with a hollow portion of the pipe 39. The holes 32b, 33b and the pipe 39 are included in the measurement flow path.

Although the second housing 32 and the third housing 33 are separate members in the present embodiment, the second housing 32 and the third housing 33 may be a single member.

The first housing 31 has recessed portions 31d, 31e. The substrate 15 is disposed in the recessed portion 31d, and the substrate 25 is disposed in the recessed portion 31e. A hole 31g is formed in the bottom surface of the recessed portion 31d, and the light emitting element 11 is provided in the hole 31g. A hole 31f is formed in the bottom surface of the recessed portion 31e, and the light emitted from the light emitting element 11 passes through the pipe 39 and the hole 31f to enter the light receiving element 21.

In the present embodiment, at least two measurement devices 1, 1A are used. The measurement device 1 is a reference measurement device that measures a reference for measuring a contamination level, and the measurement device 1A is adjusted using a measurement result of the measurement device 1. Although only the measurement device 1 is provided as the reference measurement device, the measurement device 1 can be used to adjust a plurality of measurement devices other than the measurement device 1A.

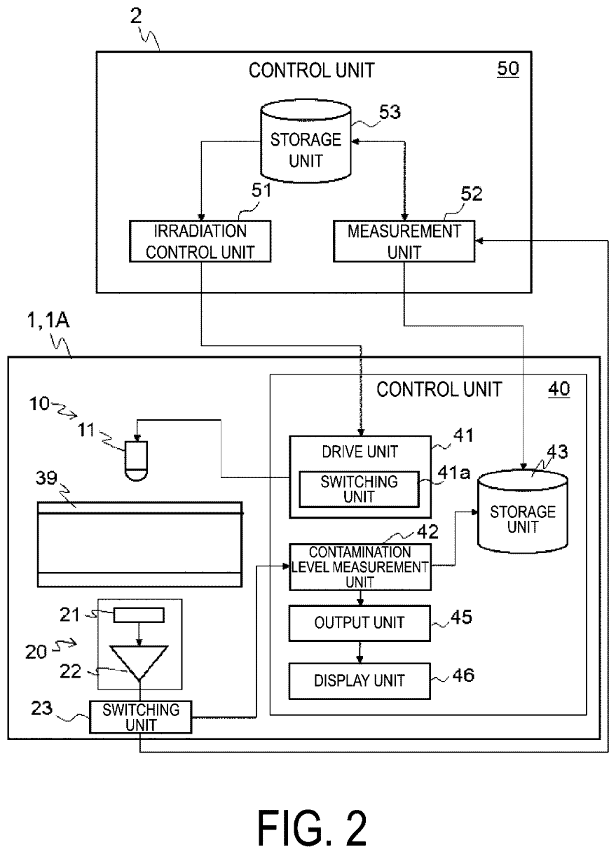

FIG. 2 is a block diagram schematically illustrating an electric configuration of the measurement device 1, 1A and an adjustment device 2 connected to the measurement device 1, 1A.

The measurement device 1, 1A includes a control unit 40, and the adjustment device 2 includes a control unit 50. The control unit 40 mainly includes a drive unit 41, a contamination level measurement unit 42, a storage unit 43, an output unit 45, and a display unit 46. The control unit 50 mainly includes an irradiation control unit 51, a measurement unit 52, and a storage unit 53.

The drive unit 41 is a functional unit that drives the light emitting element 11. The drive unit 41 includes a switching unit 41a that switches a drive mode of the light emitting element 11. The drive unit 41 includes a plurality of drive circuits that drive the light emitting element 11. A first drive circuit includes a circuit such as a constant current circuit that keeps a constant light emission amount of the light emitting element 11. When the switching unit 41a switches to the first drive circuit, the drive unit 41 causes the light emitting element 11 to continuously emit light. When the switching unit 41a switches to a second circuit that does not use the drive circuit, the drive unit 41 causes the light emitting element 11 to blink based on a pseudo signal (described in detail later) output from the irradiation control unit 51. The first drive circuit may include an APC circuit that feeds back the light reception amount of the light receiving element 21.

Although the drive unit 41 (switching unit 41a) is included in the control unit 40 in the present embodiment, the drive unit 41 (switching unit 41a) may be an analog circuit provided on the substrate 15.

The light receiving element 21 receives light emitted from the light emitting element 11 and having passed through the pipe 39. The light receiving unit 20 includes an amplifier 22, and an output signal of the light receiving element 21 is amplified by the amplifier 22 and then input to the contamination level measurement unit 42 and the measurement unit 52.

The light receiving unit 20 includes a switching unit 23 that switches the output. Here, the switching unit 23 switches whether the output signal of the light receiving element 21 is input to the contamination level measurement unit 42 or to the measurement unit 52.

Although the switching unit 23 is included in the light receiving unit 20 (substrate 25) in the present embodiment, the switching unit 23 may be included in the control unit 40.

The contamination level measurement unit 42 is a functional unit that measures the contamination level of the liquid based on the output signal from the light receiving element 21. When the oil contains particles (impurities), the amount of light blocked by the particles does not enter the light receiving element 21. The contamination level measurement unit 42 measures the amount of particles contained in the liquid, that is, the contamination level, based on the number of times and the time when the output signal from the light receiving element 21 is blocked. A known technique can be applied to handle the contamination level measurement unit 42, and the description thereof is omitted.

The contamination level measurement unit 42 is connected to the output unit 45. A display, a processing device, a storage device, a communication machine, a construction machine, and the like are connected to the output unit 45. The measurement result is displayed on a display, stored in a storage device, or output to the construction machine via a communication device and displayed on the construction machine. In the present embodiment, the display unit 46 is connected to the output unit 45. The output unit 45 may output the measurement result to an external output device or the like via a network (wired or wireless).

The irradiation control unit 51 is a functional unit that outputs a blinking signal to the drive unit 41. The pseudo signal causes the light emitting element 11 to blink (flash).

The measurement unit 52 is a functional unit that obtains an electric signal obtained by receiving light by the light receiving unit 20 when the drive unit 41 drives the light emitting element 11 using the blinking signal output from the irradiation control unit 51, thus obtaining a measurement value. The measurement unit 52 determines whether the blinking signal output from the irradiation control unit 51 is a pseudo signal based on the obtained measurement value. The processing performed by the measurement unit 52 will be described in detail later.

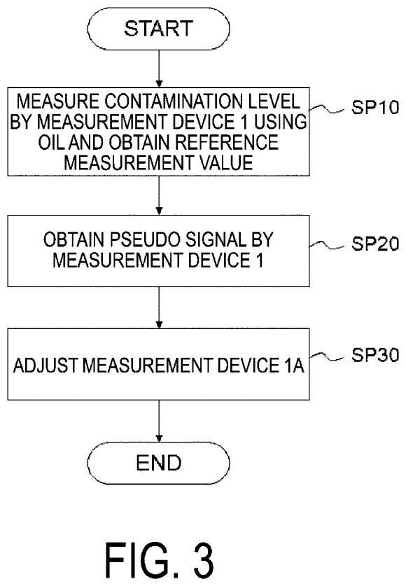

FIG. 3 is a flowchart illustrating a process flow in which the adjustment device 2 performs adjustment processing of the measurement device 1A.

### Step SP10

The measurement device 1 (corresponding to the reference measurement device of the present invention) is connected to the adjustment device 2. In a state in which oil having a given contamination level (corresponding to a first contamination level of the present invention) flows through the measurement flow path (including the pipe 39) of the measurement device 1, the drive unit 41 of the measurement device 1 emits light from the light irradiation unit 10 (corresponding to a reference light irradiation unit of the present invention) of the measurement device 1. At this time, the measurement device 1 continuously turns on the light emitting element 11. The light receiving unit 20 (corresponding to a reference light receiving unit of the present invention) of the measurement device 1 receives the emitted light, from which an electric signal is generated and obtained by the measurement unit 52, and the measurement unit 52 obtains the measurement value from the electric signal. The measurement unit 52 stores the obtained measurement value in the storage unit 53 as a reference measurement value having a given contamination level (corresponding to a first reference measurement value of the present invention). Step SP10 corresponds to a first reference value measurement step of the present invention.

The given contamination level is, for example, any one of ISO grades 16 to 22. The oil to be flowed through the pipe 39 is prepared in advance by mixing particles (dust) into clean oil so that the contamination level of the oil reaches any one of ISO grades 16 to 22. For example, dust is gradually added to oil while checking the contamination level with an adjusted measurement device, whereby the oil having the given contamination level grade is prepared.

In step SP10, the contamination level measurement unit 42 of the measurement device 1 may obtain the measurement value from the electric signal obtained by receiving the light by the light receiving unit 20, and the measurement unit 52 may obtain the measurement value.

### Step SP20

When the processing in step SP10 ends, the oil is removed from the measuring flow path of the measurement device 1 and the measurement flow path is cleaned. Then, the control unit 50 causes the light irradiation unit 10 of the measurement device 1 to emit light using a blinking signal for causing the light irradiation unit 10 of the measurement device 1 to blink in the state in which no oil is present in the measurement flow path, and sets the blinking signal when the electric signal equivalent to the reference measurement value is obtained by the light receiving unit 20 of the measurement device 1 as the pseudo signal. Step SP20 corresponds to a first pseudo signal obtaining step of the present invention.

In the present embodiment, the state in which no oil is present in the measurement flow path is a state in which no liquid flows through the measurement flow path and air is put in the measurement flow path (including the pipe 39).

### Step SP30

When the processing in steps SP10 and SP20 (corresponding to the pseudo signal generating step of the present invention) ends, a measurement device 1A (corresponding to the measurement device of the present invention) is connected to the adjustment device 2. The irradiation control unit 51 outputs the pseudo signal obtained in step SP20 to the drive unit 41. The drive unit 41 of the measurement device 1 emits light from the light irradiation unit 10 (corresponding to the light irradiation unit of the present invention) of the measurement device 1A using the pseudo signal output from the irradiation control unit 51 in a state in which no liquid flows through the measurement flow path of the measurement device 1A. That is, the pseudo signal is a signal that emits light in a state, in which no oil is present in the measurement flow path, equivalent to the light input to the light receiving unit 20 when the oil having the given contamination level (first contamination level) flows through the measurement flow path. Therefore, in step SP30, the measurement device 1A causes the light emitting element 11 to blink.

The emitted light is received by the light receiving unit 20 (corresponding to the light receiving unit of the present invention) of the measurement device 1A, from which the electric signal is obtained by the light receiving unit 20, and the measurement unit 52 obtains the electric signal. The measurement unit 52 obtains a measurement value from the obtained electric signal, and stores the measurement value in the storage unit 43 as an output value for the given contamination level in the measurement device 1A. The step SP30 corresponds to a first adjustment step of the present invention. Thus, the adjustment processing of the measurement device 1A for the given contamination level ends.

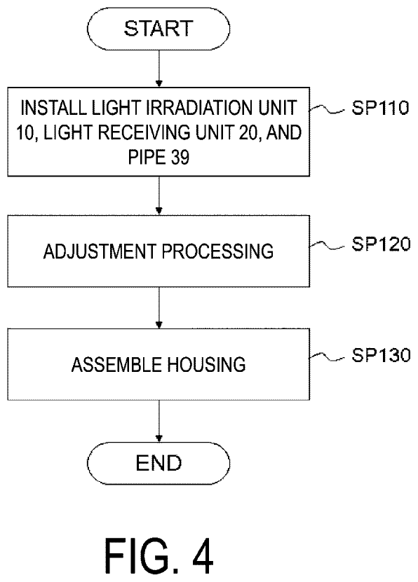

FIG. 4 is a flowchart illustrating a process flow of a method for manufacturing the measurement device 1A.

### Step SP110

First, the light irradiation unit 10 and the light receiving unit 20 are provided in the first housing 31 so as to sandwich the pipe 39. At this stage, the housing 30 is not assembled.

### Step SP120

Subsequently, the adjustment processing (step SP120) of the measurement device 1A is performed. The step SP120 and the step SP30 are the same processing.

### Step SP130

Subsequently, the housing 30 in which the pipe 39, the light irradiation unit 10, and the light receiving unit 20 are provided therein is assembled to obtain a completed product of the measurement device 1A.

The completed product of the measurement device 1A obtained in this manner is packed and shipped. Subsequently, the measurement device is provided in an device such as a construction machine or a hydraulic device, and measures the contamination level of the oil based on the output value stored in the storage unit 43 in step SP120 (step SP30) and the measurement value obtained using the electric signal obtained by the light receiving unit 20.

The processing in steps SP10 to SP30 are described in detail. First, how to obtain the measurement value using the electric signal obtained by the light receiving unit 20 at the time of measuring the contamination level in step SP10 and after shipment is described. The method for obtaining the measurement value is the same at the time of measuring the contamination level in step SP10 and after shipment.

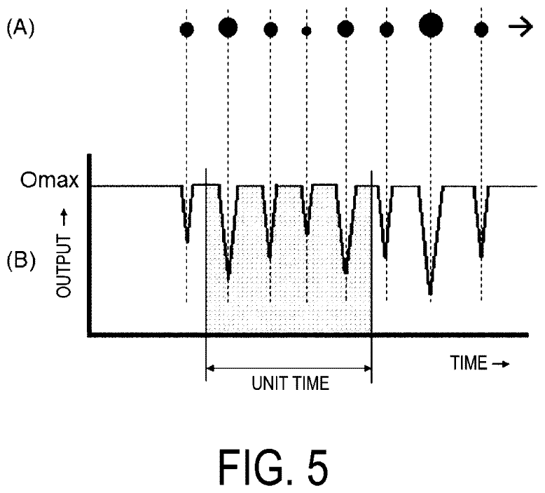

FIG. 5 is a schematic diagram for explaining the method for obtaining the measurement value, in which (A) schematically illustrates a flow of particles (dust) contained in oil flowing through the pipe 39, and (B) schematically illustrates a temporal change in the output of the electric signal.

The light irradiation unit 10 continuously emits light, and the light receiving unit 20 continuously receives the continuously emitted light. When dust passes through the pipe 39 (see FIG. 5(A)), a shadow is formed, and thus the amount of light received by the light receiving unit 20 decreases, and the output decreases as compared with the case in which no dust is present (see FIG. 5(B)).

The contamination level measurement unit 42 and the measurement unit 52 obtain the integrated value per unit time of the electric signal illustrated in FIG. 5(B) (see the hatching portion in FIG. 5(B)). The contamination level measurement unit 42 and the measurement unit 52 obtain a moving average of the integrated value per unit time of the electric signal and smooth the integrated value per unit time. When measuring the contamination level of oil containing dust, the level of output reduction varies depending on the size of the dust and the like, and therefore, it is necessary to smooth the integrated value per unit time.

The contamination level measurement unit 42 calculates a total dust amount (contamination level grade) per unit time based on the smoothed integrated value. The measurement unit 52 obtains a reference measurement value based on the smoothed integrated value.

The light emitting element 11 and the light receiving element 21 have individual differences. For example, there is an individual difference in the light emission efficiency of the light emitting element 11, and a variation occurs in the amount of light to be irradiated. There is also an individual difference in the conversion efficiency from light to voltage in the light receiving element 21, and a variation occurs in the output value. Therefore, when no dust passes through the pipe 39, the output value obtained from the light receiving unit 20 is different even when the same voltage is applied to the light emitting element 11.

Therefore, when measuring the contamination level in step SP10 and after shipment, the drive unit 41 and the irradiation control unit 51 adjust the power level (brightness of the light emitting element 11) applied to the light irradiation unit 10, so that a maximum value Omax (see FIG. 5(B)) of the electric signal obtained by the light receiving unit 20 can be a predetermined value, thus adjusting the output level of the output obtained from the light receiving unit 20.

The contamination level measurement unit 42 and the measurement unit 52 may adjust the output level by processing the electric signal output from the sensing circuit by also considering the individual differences in the electronic circuit (sensing circuit).

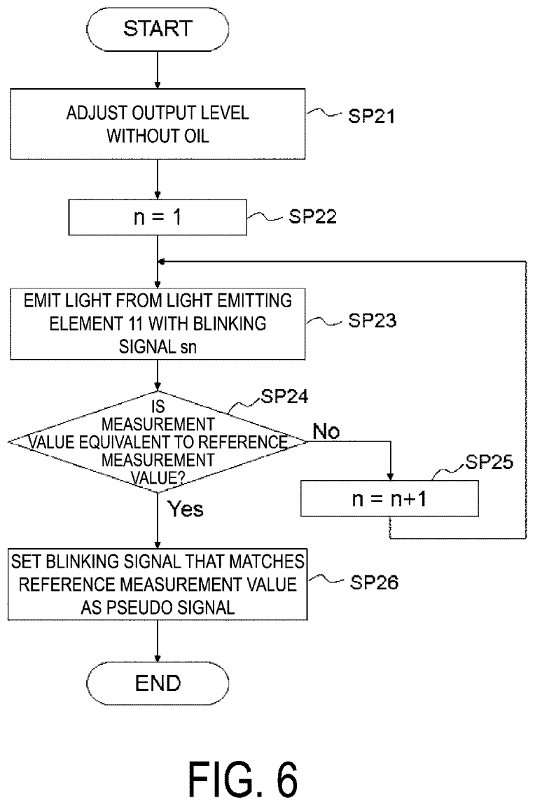

FIG. 6 is a flowchart illustrating a process flow for obtaining the pseudo signal (step SP20). First, the measurement device 1 is removed from the adjustment device 2, and the measurement device 1A is connected to the adjustment device 2.

### Step SP21

The measurement flow path of the measurement device 1 is cleaned by removing the oil therefrom to attain a state in which no oil is present in the measurement flow path (here, a state in which air is present in the pipe 39). The irradiation control unit 51 adjusts the power applied to the light irradiation unit 10, that is, the output level of the light irradiation unit 10, so that the maximum value Omax of the electric signal obtained by the light receiving unit 20 becomes equivalent to the maximum value Omax in step SP10.

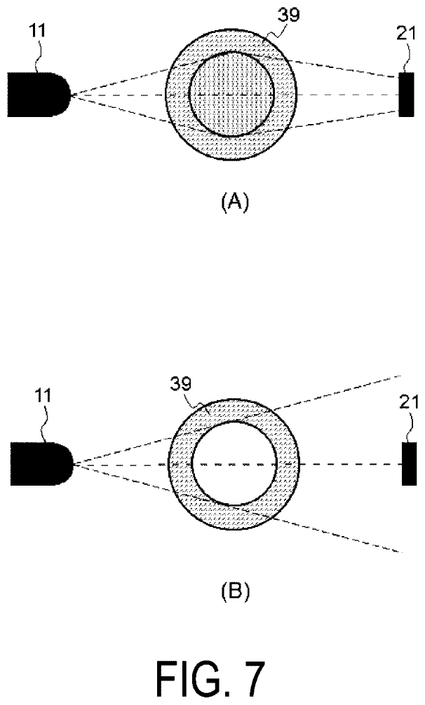

FIG. 7 illustrates a state of light emitted from the light emitting element 11, in which (A) illustrates a state in which oil is present in the pipe 39, and (B) illustrates a state in which no oil is present and air is present in the pipe 39. A refractive index of the oil is 1.467, and in a state in which the oil is present in the pipe 39, the pipe 39 including oil acts as a lens, and the light emitted from the light emitting element 11 is focused on the light receiving element 21 (see FIG. 7(A)). In contrast, the refractive index of air is 1, so that the pipe 39 does not act as a lens in a state in which air is present in the pipe 39, and the light emitted from the light emitting element 11 is not focused on the light receiving element 21 (see FIG. 7(B)). Therefore, in the state in which air is present in the pipe 39, the amount of light entering the light receiving element 21 is small and the maximum value Omax of the electric signal obtained by the light receiving unit 20 is small, as compared with the state in which the oil is present in the pipe 39.

Therefore, in step SP21, the irradiation control unit 51 adjusts the output level of the light irradiation unit 10 so that the maximum value Omax of the electric signal obtained by the light receiving unit 20 becomes the same as the maximum value Omax in step SP10.

For example, assuming that the maximum value Omax of the electric signal obtained by the light receiving unit 20 in the state in which the oil is present in the pipe 39 is 3.5 V and the maximum value Omax of the electric signal obtained by the light receiving unit 20 in the state in which the air is present in the pipe 39 is 1 V, the irradiation control unit 51 multiplies the electric power applied to the light irradiation unit 10 by 3.5 times the state in which the air is present in the pipe 39 compared to the state in which the oil is present in the pipe 39, thus providing an equivalent maximum value Omax. This improves the accuracy of the pseudo signal.

### Steps SP22, 23

The description returns to FIG. 6. The irradiation control unit 51 outputs the blinking signal sn to the drive unit 41, and the drive unit 41 causes the light emitting element 11 to blink using the blinking signal sn. Since n=1 is set in step SP22, the blinking signal is a blinking signal s1 when step SP23 is executed first. The light receiving unit 20 receives the light emitted in this manner, and the obtained electric signal is input to the measurement unit 52. The measurement unit 52 obtains the measurement value based on the electric signal.

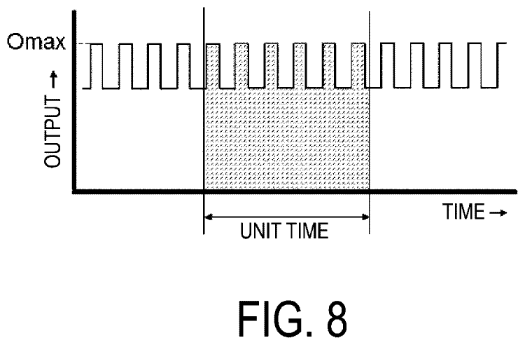

FIG. 8 schematically illustrates an electric signal obtained by the light receiving unit 20 when the light emitting element 11 blinks using the blinking signal sn. The electric signal includes a high output state and a low output state appearing periodically. The measurement unit 52 obtains an integrated value per unit time of the electric signal (see a shaded portion in FIG. 8), and sets the integrated value as a measurement value.

The description returns to FIG. 6. The storage unit 53 stores a plurality of blinking signals sn (n is a natural number). The irradiation control unit 51 obtains the blinking signal sn necessary for executing step SP23 from the storage unit 53 and outputs the blinking signal sn to the drive unit 41.

### Step SP24

The measurement unit 52 determines whether the measurement value obtained in step SP23 is equivalent to the reference measurement value obtained in step SP10. The measurement unit 52 obtains the reference measurement value from the storage unit 53. Here, being substantially equivalent to the reference measurement value refers to a concept of being substantially equivalent to the reference measurement value, that is, matching the reference measurement value or not matching the reference measurement value but an error is very small.

When the measurement value obtained in step SP23 is not equivalent to the reference measurement value (No in step SP24), the process proceeds to step SP25. When the measurement value obtained in step SP23 is equal to the reference measurement value (Yes in step SP24), the process proceeds to step SP26.

### Step SP25

When the measurement value obtained in step SP23 is not equivalent to the reference measurement value (No in step SP24), the measurement unit 52 sets n=n+1 and returns the process to step SP23. For example, when the measurement value obtained when the light emitting element 11 is driven to blink using the blinking signal s1 is not equivalent to the reference measurement value, the measurement unit 52 performs the processing in step SP23 using the blinking signal s2. In this way, steps SP23 to SP25 are repeated until the blinking signal sn equivalent to the reference measurement value is found.

### Step SP26

When the measurement value obtained in step SP23 is equivalent to the reference measurement value (Yes in step SP24), the measurement unit 52 sets the blinking signal sn used when the measurement value is equivalent to the reference measurement value as the pseudo signal (corresponding to the first pseudo signal of the present invention). Then, the measurement unit 52 stores the pseudo signal in the storage unit 53. Thus, a series of processing steps in step SP20 end. The accurate pseudo signal can be obtained by making the measurement value, which is the integrated value per unit time, equivalent to the reference measurement value. This improves the adjustment accuracy.

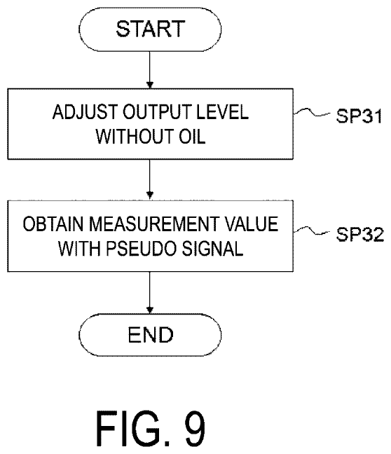

FIG. 9 is a flowchart illustrating a process flow of step SP30.

### Step SP31

In the state in which no oil is present in the measurement flow path of the measurement device 1A (here, the state in which air is present in the pipe 39), the irradiation control unit 51 adjusts the output level of the light irradiation unit 10 based on the adjustment result of the output level in step SP21. For example, the irradiation control unit 51 applies the same power as the power applied to the light irradiation unit 10 by the irradiation control unit 51 in step SP21 to the light irradiation unit 10 via the drive unit 41. Accordingly, the maximum value Omax of the electric signal obtained by the light receiving unit 20 becomes equivalent to the maximum value Omax of the electric signal obtained by the light receiving unit 20 when the state in which the oil is present in the measurement flow path without adjusting the output level.

### Step SP32

The irradiation control unit 51 outputs the pseudo signal stored in the storage unit 53 to the drive unit 41. The drive unit 41 emits light from the light irradiation unit 10 using the pseudo signal after the output level is adjusted in step SP31. The light receiving unit 20 receives the light emitted in this manner, and the obtained electric signal is input to the measurement unit 52. The measurement unit 52 obtains a measurement value (corresponding to the measurement value in the first contamination level of the present invention) based on the electric signal. For example, the measurement unit 52 obtains the integrated value per unit time of the electric signal as in step SP23, and sets the integrated value as the measurement value. The measurement unit 52 stores the measurement value in the storage unit 43. Thus, a series of processing steps in step SP30 end.

According to the present embodiment, the measurement device 1A can be adjusted without flowing the oil through the measurement flow path including the pipe 39. This reduces the time required for the adjustment. This also improves the adjustment accuracy.

When the adjustment is performed by pouring oil into the measurement flow path as in the related art, it takes time and effort to clean the measurement flow path after the adjustment, and it takes time to complete the adjustment. In some cases, air transport is used for delivery, but since air transporters dislike oil remaining inside the transported items, packaging costs increase if there is a history of oil being poured into the measurement flow path.

When adjusting the measurement device 1A by pouring oil into the measurement flow path, it is necessary to prepare oil containing dust. However, heating the oil and dispersing the dust in the oil takes time. In particular, to disperse the dust in the oil, the dust should be added in multiple batches, and should continue to be added until the desired contamination level is achieved, thus taking a long time. In order to reuse the oil that has been used for adjustments, it should be cleaned, which also takes time.

In contrast, adjusting the measurement device 1A without flowing oil through the measurement flow path, as in the present embodiment, eliminates the time, cost, and labor associated with the use of oil.

When using the oil containing dust during adjustment of the measurement device 1A, it takes time for the contamination level measurement unit 42 to execute measurement. In contrast, the adjustment can be performed quickly when using the pseudo signal because no smoothing is needed.

The oil with a known contamination level can be prepared by mixing particles within a predetermined particle size into oil in a predetermined amount. However, variations in particle size and mixing amount cannot be eliminated, which may result in instability in the adjustment accuracy. In contrast, the present embodiment uses the pseudo signal, which stabilizes the light emitted from the light irradiation unit 10 and improves the adjustment accuracy.

According to the present embodiment, the output level of the light irradiation unit 10 is adjusted prior to measurement, which allows for obtaining the accurate pseudo signal and performing the accurate adjustment.

In the present embodiment, the pseudo signal is obtained and the measurement device 1A is adjusted in the state in which no oil flows through the measurement flow path and air is present in the pipe 39. However, the absence of oil in the measurement flow path is not limited to this case. For example, the state in which no oil is present in the measurement flow path may be a state in which air is contained in the measurement flow path without flowing a liquid, a state in which a liquid other than oil flows through the measurement flow path, or a state in which a glass rod is provided instead of the pipe 39.

The state in which a liquid other than oil flows through the measurement flow path may be a state in which water, alcohol, or a mixture of water and alcohol is put in the measurement flow path including the pipe 39. The refractive index of air is 1, but the refractive indices of water and alcohol are 1.3 to 1.4, which are close to the refractive index of oil of 1.467. Specifically, the refractive index of water is 1.33, the refractive index of ethanol is 1.361, the refractive index of methanol is 1.329, and the refractive index of isopropyl alcohol is 1.384.

Thus, in a state in which water, alcohol, or a mixture of water and alcohol is put in the pipe 39, the pipe 39 acts as a lens, and light emitted from the light emitting element 11 is focused on the light receiving element 21, unlike the state in which air is put in the pipe 39. This facilitates obtaining of the pseudo signal and adjustment of the measurement device 1A. Depending on the type of liquid flowing through the pipe 39, the pseudo signal can be obtained and the measurement device 1A can be adjusted without adjusting the output level (steps SP21 and SP31 are not essential). Because water and alcohol dry quickly, any water or alcohol poured into the measurement flow path dries quickly as well, so that time required for adjustment is shortened.

For example, a glass rod can be installed instead of the pipe 39 to achieve the state in which no oil is present in the measurement flow path. The refractive index of glass, such as quartz glass, is from 1.46 to 1.47, and the refractive index of BK7 is from 1.51 to 1.53, both of which being close to 1.467 which is the refractive index of oil. Therefore, the glass rod acts as a lens, and the light emitted from the light emitting element 11 is focused on the light receiving element 21. This facilitates obtaining of the pseudo signal and adjustment of the measurement device 1A. The obtaining of the pseudo signal and the adjustment of the measurement device 1A without adjusting the output level (steps SP21 and SP31 are not necessary).

In the present embodiment, the reference measurement value (first reference measurement value) is measured using the oil having the given contamination level (first contamination level) in step SP10, the signal that emits light equivalent to the light input to the light receiving unit 20 when the oil having the first contamination level flows through the measurement flow path in the state in which no oil is present in the measurement flow path is obtained as the pseudo signal in step SP20, and the measurement for the first contamination level is obtained using the pseudo signal, that is, the adjustment of the measurement device 1A is performed for one contamination level in step SP30. However, the adjustment of the measurement device 1A may also be performed for a plurality of contamination levels (at least two, including the first and second contamination levels). For example, when the ISO grade of the given contamination level is from 16 to 22, the first contamination level may be ISO grade 16, and the second contamination level may be ISO grade 20.

That is, the control units 40, 50 each search for the reference measurement value and the pseudo signal for each grade for which the measurement value is desired to be obtained. The control units 40, 50 each obtain the measurement value using the pseudo signal in each grade. For example, when adjusting the measurement device 1A for the first and second contamination levels, the reference measurement values (the first and second reference measurement values) for the first and second contamination levels can be measured in step SP10, the first and second pseudo signals respectively corresponding to the first and second contamination levels (the first and second pseudo signals) can be obtained in step SP20, and then, in step SP30, the first and second pseudo signals can be used to obtain the measurement values for the first and second contamination levels, respectively.

In the present embodiment, one measurement device 1A is adjusted, but a plurality of measurement devices 1A can be successively adjusted. In this case, the processing in step SP30 can be performed successively for the number of measurement devices 1A.

In the present embodiment, steps SP10 and SP20 are performed prior to step SP30, but steps SP10 and SP20 are not essential. For example, the pseudo signal may be stored in the storage unit 43 or 53 in advance, and the processing in step SP30 may be performed using the pseudo signal. In this case, the processing for obtaining the pseudo signal is not limited to the method in steps SP10 and SP20. However, in order to increase the accuracy of the pseudo signal, it is desirable to obtain the pseudo signal using the method in steps SP10 and SP20.

### Second Embodiment

In the first embodiment, the measurement device 1A is adjusted using the pseudo signal, but the method for adjusting the measurement device 1A is not limited to this. A second embodiment of the present invention is an embodiment in which the measurement device 1A is adjusted using a rod-shaped member provided with a plurality of light-blocking slits that do not transmit light. The second embodiment is described below. The same or similar configurations as those in the first embodiment are denoted by the same reference signs, and description thereof is omitted.

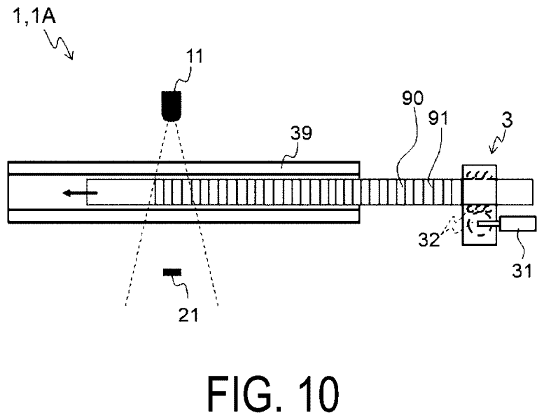

FIG. 10 schematically illustrates a state in which a pseudo signal is obtained by the measurement device 1 and a measurement value is obtained by the measurement device 1A. The light irradiation unit 10 continuously turns on light while moving the rod-shaped member 90 in the measurement flow path (including the pipe 39). The light receiving unit 20 receives the light emitted from the light irradiation unit 10 and having passed through the pipe 39 and the rod-shaped member 90 to obtain the electric signal. The rod-shaped member 90 is, for example, a glass rod, and a plurality of light-blocking slits 91 that do not transmit light are provided in the rod-shaped member 90.

In the embodiment illustrated in FIG. 10, the light-blocking slits 91 are provided at regular intervals. However, the shape of the rod-shaped member 90 is not limited to this. Any width and spacing of the light-blocking slits 91 can be set. For example, a plurality of types of light-blocking slits having different thicknesses may be provided in the rod-shaped member 90. The intervals between adjacent light-blocking slits 91 may not be the same.

The rod-shaped member 90 is provided with the drive device 3. The drive device 3 mainly includes a drive unit 31 such as an actuator that moves the rod-shaped member 90, and a transmission unit 32 that transmits an output of the drive unit 31 to the rod-shaped member 90. The drive unit 31 moves the rod-shaped member 90 along the longitudinal direction of the rod-shaped member 90 via the transmission unit 36.

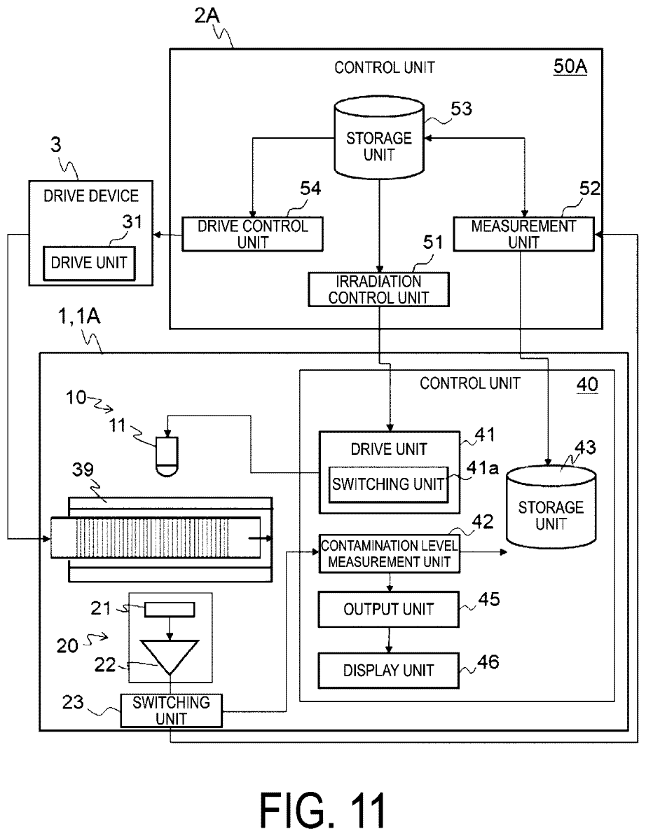

FIG. 11 is a block diagram schematically illustrating an electrical configuration of the measurement device 1, 1A, and the adjustment device 2A connected to the measurement device 1, 1A.

The measurement device 1, 1A includes the control unit 40 and the adjustment device 2A includes the control unit 50A. The control unit 50A mainly includes an irradiation control unit 51, the measurement unit 52, the storage unit 53, and a drive control unit 54.

The drive control unit 54 is a functional unit that outputs a signal for driving the drive unit 35 to the drive device 3. The signal sm for driving the drive unit 35 moves the rod-shaped member bn at a predetermined speed cm. A plurality of signals sm (m is a natural number) are stored in the storage unit 53.

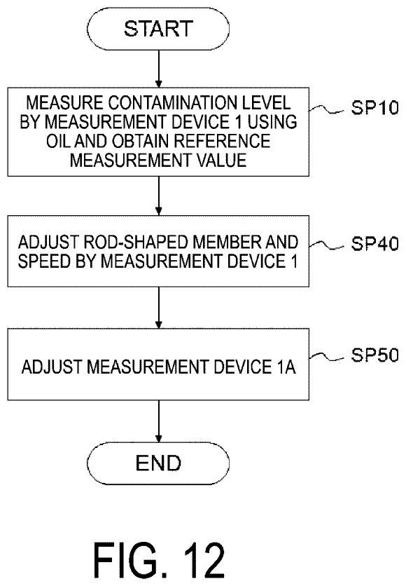

FIG. 12 is a flowchart illustrating a process flow for adjusting the measurement device 1A by the adjustment device 2A.

### Step SP10

The measurement device 1 is connected to the adjustment device 2A. In a state in which oil having a given contamination level flows through the measurement flow path of the measurement device 1, the drive unit 41 of the measurement device 1 continuously emits light from the light irradiation unit 10 of the measurement device 1. The light receiving unit 20 of the measurement device 1 receives the light emitted in this manner to obtain an electric signal which is measured and obtained by the measurement unit 52. The measurement unit 52 stores the obtained measurement value in the storage unit 53 as a reference measurement value for a given contamination level. Step SP40 corresponds to the first reference value measurement step of the present invention.

### Step SP40

When the processing in step SP10 ends, the oil is removed from the measuring flow path of the measurement device 1 and the measurement flow path is cleaned. A plurality of rod-shaped members 90 having different widths and numbers of light-blocking slits 91 are prepared. The plurality of rod-shaped members will be referred to as rod-shaped members bn (n is a natural number) below.

The control unit 50A adjusts the size and number of the light-blocking slits 91 of the rod-shaped member 90 and the speed of moving the rod-shaped member 90 in the state in which no oil is present in the measurement flow path. For example, in the state in which light is continuously emitted from the light irradiation unit 10 of the measurement device 1, the light receiving unit 20 of the measurement device 1 receives the light and obtains electric signals while moving the rod-shaped member bn at a given speed. The control unit 50A obtains the rod-shaped member bn and the moving speed thereof (the first speed of the present invention) when the light receiving unit 20 of the measurement device 1 obtains the electric signal equivalent to the reference measurement value. Step SP40 corresponds to the adjustment step of the present invention.

### Step SP50

When the processing in steps SP10 and SP40 (corresponding to the rod-shaped member adjustment step of the present invention) ends, the measurement device 1A is connected to the adjustment device 2A. The drive unit 41 of the measurement device 1 continuously emits light from the light irradiation unit 10 of the measurement device 1A. At this time, the rod-shaped member bn is moved in the pipe 39 at the moving speed obtained in step SP40.

The light receiving unit 20 of the measurement device 1A receives the light emitted in this manner, and the obtained electric signal is measured and obtained by the measurement unit 52. The measurement unit 52 stores the obtained measurement value in the storage unit 43 as the output value for the given contamination level in the measurement device 1A. Step SP50 corresponds to the first adjustment step of the present invention. Thus, the adjustment processing of the measurement device 1A for the given contamination level ends.

A method for manufacturing the measurement device 1A in the present embodiment is the same as that in the first embodiment. That is, the light irradiation unit 10 and the light receiving unit 20 are provided in the first housing 31 so as to sandwich the pipe 39 (step SP110), the measurement device 1A is adjusted (step SP120), and the housing 30 in which the pipe 39, the light irradiation unit 10, and the light receiving unit 20 are provided therein is assembled to obtain a completed product of the measurement device 1A (step SP130). The step SP120 in the present embodiment is the same processing as the step SP50.

The completed product of the measurement device 1A obtained in this manner is packed and shipped. Subsequently, the measurement device is installed in construction machinery, hydraulic equipment, or other devices, and measures the contamination level of the oil based on the output value stored in the storage unit 43 in step SP120 (step SP50) and the measurement value obtained using the electric signal obtained by the light receiving unit 20.

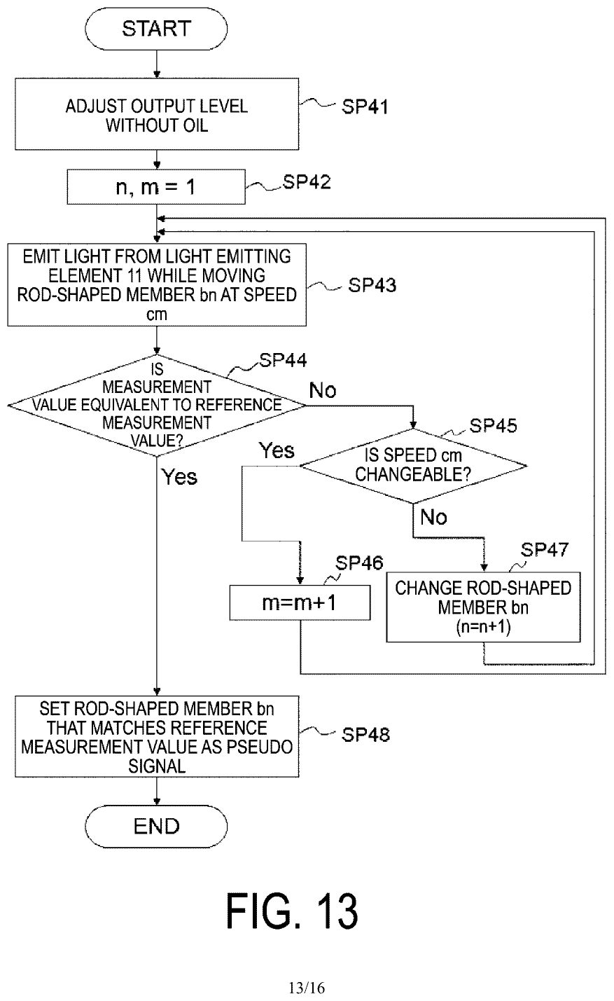

FIG. 13 is a flowchart illustrating a process flow for an adjustment step (step SP40). First, the measurement device 1 is removed from the adjustment device 2A, and the measurement device 1A is connected to the adjustment device 2A. A plurality of rod-shaped members bn (n is a natural number) are prepared in advance.

### Step SP41

Oil is removed from the measurement flow path of the measurement device 1, and the measurement flow path is cleaned to achieve a state in which air is put into the pipe 39 of the measurement flow path. The irradiation control unit 51 adjusts the power applied to the light irradiation unit 10, that is, the output level of the light irradiation unit 10, so that the maximum value Omax of the electric signal obtained by the light receiving unit 20 becomes equivalent to the maximum value Omax in step SP10. The processing in step SP41 is similar to step SP21.

### Steps SP42, 43

The irradiation control unit 51 obtains a speed cm from the storage unit 53 and outputs it to the drive unit 41, thereby moving the rod-shaped member bn (a provisional rod-shaped member of the present invention) at the speed cm (a provisional speed of the present invention) via the drive device 3. In this state, the drive unit 41 continuously turns on the light irradiation unit 10 (the light emitting element 11). The light receiving unit 20 receives the light emitted in this manner, and the obtained electric signal is input to the measurement unit 52. The measurement unit 52 obtains the measurement value based on the electric signal. The measurement unit 52 obtains the integrated value of the electric signal per unit time, and sets the integrated value as the measurement value.

To set n, m=1 in step SP42, the rod-shaped member when the step SP43 is first executed is the rod-shaped member b1, and the speed is the speed c1.

### Step SP44

The measurement unit 52 determines whether the measurement value obtained in step SP43 is equivalent to the reference measurement value obtained in step SP10. The measurement unit 52 obtains the reference measurement value from the storage unit 53.

When the measurement value obtained in step SP43 is not equivalent to the reference measurement value (No in step SP44), the process proceeds to step SP45. When the measurement value obtained in step SP23 is equivalent to the reference measurement value (Yes in step SP44), the process proceeds to step SP48.

### Steps SP45 to SP47

When the measurement value obtained in step SP43 is not equivalent to the reference measurement value (No in step SP44), the measurement unit 52 determines whether the speed cm is changeable, that is, whether all the speeds stored in the storage unit 53 have been executed (step SP45).

When the speed cm is changeable (Yes in step SP45), the measurement unit 52 sets m=m+1 (step SP46) and returns the process to step SP43. When the speed cm is not changeable (No in step SP45), the measurement unit 52 returns m to 1, changes the rod-shaped member bn assuming n=n+1 (step SP47), and returns the process to step SP43. In this way, steps SP43 to SP47 are repeated until the rod-shaped member bn and the speed cm at which the measurement value is equivalent to the reference measurement value are found. Step SP48

When the measurement value obtained in step SP43 is equivalent to the reference measurement value (Yes in step SP44), the measurement unit 52 sets the rod-shaped member bn and the speed cm (hereinafter, the rod-shaped member bx and the speed cx) used when the measurement value is equivalent to the reference measurement value as the rod-shaped member (the rod-shaped member of the present invention) and the speed (the first speed of the present invention) for a given contamination level (the first contamination level). Then, the measurement unit 52 stores the rod-shaped member bx and the speed cx in the storage unit 53. Thus, a series of processing in steps SP40 ends.

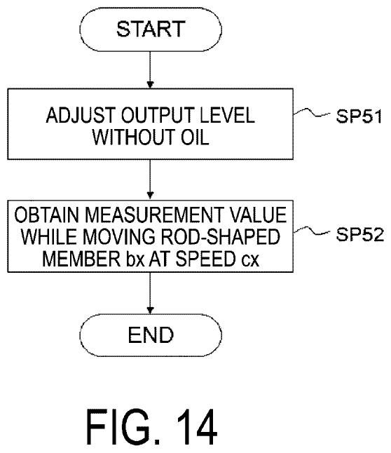

FIG. 14 is a flowchart illustrating the process flow of the processing in step SP50.

### Step SP51

In the state in which no oil is present in the measurement flow path of the measurement device 1A (here, the state in which air is present in the pipe 39), the irradiation control unit 51 adjusts the output level of the light irradiation unit 10 based on the adjustment result of the output level in step SP21. The processing in step SP51 is similar to that in step SP31.

### Step SP52

The drive control unit 54 outputs the speed cx stored in the storage unit 53 to the drive device 3, and the drive unit 35 moves the rod-shaped member bx at the speed cx. The drive unit 41 and the irradiation control unit 51 continuously emits light from the light irradiation unit 10 after the output level is adjusted in step SP31. The light receiving unit 20 receives the light emitted in this manner, and the obtained electric signal is input to the measurement unit 52. The measurement unit 52 obtains a measurement value (corresponding to the measurement value in the first contamination level of the present invention) based on the electric signal. For example, the measurement unit 52 obtains the integrated value per unit time of the electric signal as in step SP43, and sets the integrated value as the measurement value.

According to the present embodiment, the measurement device 1A can be adjusted without flowing the oil through the measurement flow path including the pipe 39. Therefore, the time required for adjustment can be shortened. This also improves the adjustment accuracy.

According to the present embodiment, the rod-shaped member 90 is made of glass, so that the light can be focused on the light receiving unit 20 as in the case in which the oil flows through the pipe 39. This makes it possible to obtain the pseudo signal and adjust the measurement device 1A without adjusting the output level (steps SP41 and SP51 are not essential).

According to the present embodiment, the output level of the light irradiation unit 10 is adjusted before obtaining the measurement value, whereby the accurate pseudo signal can be obtained, and the adjustment can be performed accurately.

In the present embodiment, the adjustment of the measurement device 1A is performed for a single contamination level, but the adjustment of the measurement device 1A may be performed for a plurality of contamination levels (at least two of the first contamination level and the second contamination level) as in the first embodiment. In the present embodiment, one measurement device 1A is adjusted, but a plurality of measurement devices 1A can be adjusted successively as in the first embodiment.

### Third Embodiment

The first and second embodiments each include one light emitting element 11 and one light receiving element 21, but the number of light emitting elements 11 and light receiving elements 21 is not limited to thereto. For example, a plurality of light emitting elements 11 and a plurality of light receiving elements 21 may be provided, or one light emitting element 11 and two light receiving elements 21 may be provided.

In a third embodiment of the present invention, two light emitting elements 11 and two light receiving elements 21 are provided, and the contamination level is measured based on a difference in light received by the two light receiving elements 21. The third embodiment of the present invention is described below. The configurations and processing steps similar to those in the first and second embodiments are denoted by the same reference signs, and description thereof is omitted.

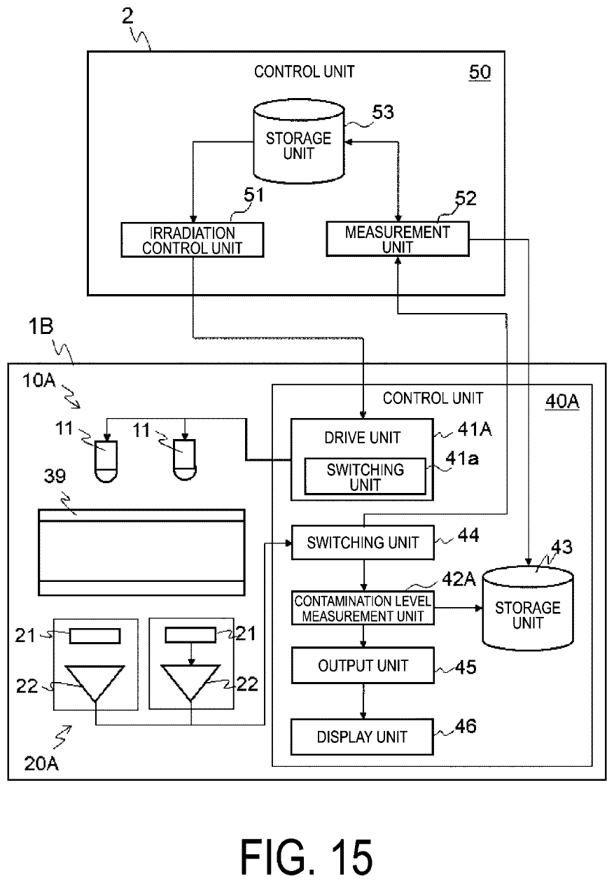

FIG. 15 is a block diagram schematically illustrating an electric configuration of a measurement device 1B and the adjustment device 2 connected to the measurement device 1B. The measurement device 1B is a measurement device to be adjusted, and the measurement device 1 is used to obtain the reference measurement value and the pseudo signal.

The measurement device 1B includes a light irradiation unit 10A including a plurality of (here, two) light emitting elements 11, and a light receiving unit 20A including a plurality of (here, two) light receiving elements 21 and amplifiers 22. The measurement device 1B also includes a control unit 40A, and the adjustment device 2 includes the control unit 50. The control unit 40A mainly includes a drive unit 41A, a contamination level measurement unit 42A, the storage unit 43, a switching unit 44, the output unit 45, and the display unit 46.

The drive unit 41A is a functional unit that drives only a desired light emitting element 11 of the two light emitting elements 11. Driving the light emitting element 11 is similar to that of the drive unit 41, and thus the description thereof is omitted.

The contamination level measurement unit 42A is a functional unit that measures the contamination level of the liquid based on the signals output from the two light receiving elements 21. For example, the contamination level measurement unit 42A measures the contamination level based on the difference between the measurement value obtained from the signal output from one of the light receiving elements 21 and the measurement value obtained from the signal output from the other of the light receiving elements 21. Since various methods can be used for the measurement of the contamination level, the description thereof is omitted.

The switching unit 44 is a functional unit that switches the output from the light receiving unit 20A. The switching unit 44 outputs the signal from the light receiving unit 20A to the measurement unit 52 during adjustment, and outputs the signal from the light receiving unit 20A to the contamination level measurement unit 42 during measurement of the contamination level after shipment. In this way, the switching unit 44 changes the destination of the light from the light receiving unit 20A depending on whether the adjustment is performed or the contamination level is measured.

The switching unit 44 separately outputs the signals from the plurality of light receiving elements 21 and the amplifier 22 to the measurement unit 52 at the time of adjustment. For example, the switching unit 44 outputs the signal obtained by the light receiving element 21 corresponding to the driven light emitting element 11 to the measurement unit 52.

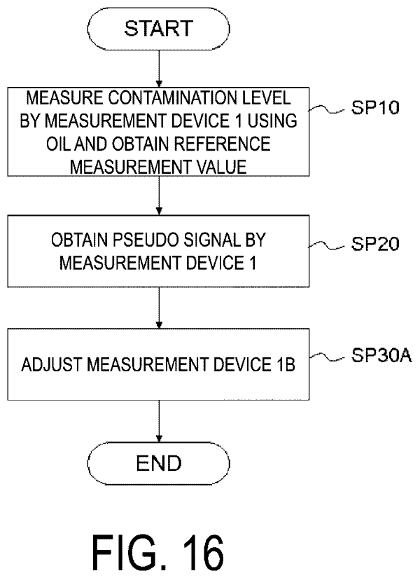

FIG. 16 is a flowchart illustrating the process flow in which the adjustment device 2 adjusts the measurement device 1B.

### Step SP10

The measurement device 1 is connected to the adjustment device 2. In the state in which the oil having the given contamination level (first contamination level) flows through the measurement flow path of the measurement device 1, the drive unit 41 of the measurement device 1 continuously emits light from the light irradiation unit 10 of the measurement device 1. The light receiving unit 20 of the measurement device 1 receives the light emitted in this manner to obtain an electric signal which is measured and obtained by the measurement unit 52. The measurement unit 52 stores the obtained measurement value in the storage unit 53 as the reference measurement value (first reference measurement value) for the given contamination level.

### Step SP20

When the processing in step SP10 ends, the oil is removed from the measuring flow path of the measurement device 1 and the measurement flow path is cleaned. Then, the control unit 50 causes the light irradiation unit 10 of the measurement device 1 to emit light using a blinking signal for causing the light irradiation unit 10 of the measurement device 1 to blink in the state in which no oil is present in the measurement flow path, and sets the blinking signal when the electric signal equivalent to the reference measurement value is obtained by the light receiving unit 20 of the measurement device 1 as the pseudo signal.

### Step SP30A

When the processing in step SP20A ends, the measurement device 1B (corresponding to the measurement device of the present invention) is connected to the adjustment device 2. The irradiation control unit 51 outputs the pseudo signal obtained in step SP20A to the drive unit 41A. The drive unit 41A of the measurement device 1 drives any of the light emitting elements 11 of the light irradiation unit 10A (corresponding to the light emitting unit of the present invention) of the measurement device 1B using the pseudo signal output from the irradiation control unit 51 in a state in which no liquid flows through the measurement flow path of the measurement device 1A. The emitted light is received by the light receiving elements 21 corresponding to the light emitting elements 11 having emitted the light in the light receiving unit 20A (corresponding to the light receiving unit of the invention) of the measurement device 1B, and the obtained electric signal is received by the measurement unit 52 via the switching unit 44. The measurement unit 52 obtains the measurement value based on the electric signal, and stores the measurement value in the storage unit 43 as an output value at a given contamination level in the measurement device 1A. Step SP30A corresponds to a first adjustment step of the present invention.

The difference between steps SP30 and SP30A is that the measurement values are obtained repeatedly for the number of light emitting elements 11 and light receiving elements 21 (in this case, twice), while the specific processing steps are the same. Thus, the adjustment processing of the measurement device 1B at a given contamination level ends.

The method for manufacturing the measurement device 1B is the same as the method for manufacturing the measurement device 1A (FIG. 4).

According to the present embodiment, the adjustment of the measurement device 1B can be performed without flowing oil through the measurement flow path including the pipe 39. Therefore, the time required for adjustment can be shortened. This also improves the adjustment accuracy.

In the present embodiment, the adjustment is performed in the state in which air is present in the pipe 39. However, as in the first embodiment, the adjustment may be performed in the state in which water, alcohol, or a mixture of water and alcohol is present in the measurement flow path including the pipe 39, or in the state in which the glass rod is provided instead of the pipe 39. In the present embodiment, the adjustment is performed using the pseudo signal, but the adjustment may be performed using the rod-shaped member 90 as in the second embodiment.

In the present embodiment, steps SP10 and 20 are performed using the measurement device 1 having one light emitting element 11 and one light receiving element 21, but the steps SP10 and 20 may be performed using a measurement device having a plurality of light emitting elements 11 and a plurality of light receiving elements 21. In this case, the processing in steps SP10 and 20 may be repeated by the number of the light emitting elements 11 and the light receiving elements 21 (here, twice), and the specific processing details are the same.

The embodiments of the present invention have been described above in detail with reference to the drawings, but specific configurations are not limited to the embodiments and changes in design or the like without departing from the gist of the invention are also included. For example, the examples described above are described in detail to facilitate understanding of the present invention, and are not necessarily limited to those including all the configurations described above. In addition, the configuration of one embodiment can be replaced with the configurations of other embodiments, and addition, deletion, replacement, or the like of other configurations can be made on the configuration of the one embodiment.

In the present invention, “substantially” refers to a concept that includes not only cases of being strictly identical, but also includes errors and deformations that do not lose their identity. For example, “substantially orthogonal” refers to a concept that is not limited to the case of being strictly orthogonal, but also includes errors of a few degrees, for example. Simple expressions such as orthogonal, parallel, and identical are not to be understood as merely strictly orthogonal, parallel, and identical, for example, but also include cases that are substantially parallel, substantially orthogonal, or substantially identical.

In the present invention, “vicinity” means a region that includes a range (which can be defined arbitrarily) near a reference position. For example, “vicinity of an end” refers to a concept that indicates a range of region in the vicinity of the end that may or may not include the end itself.

### REFERENCE SIGNS LIST

1, 1A, 1B Measurement device
2, 2A Adjustment device
3 Drive device
10, 10A Light irradiation unit
11 Light emitting element
15 Substrate
20, 20A Light receiving unit
21 Light receiving element
22 Amplifier
23 Switching unit
25 Substrate
30 Housing
31 First housing
31a Bore
31b Hole
31c Female thread portion
31d, 31e Recessed portion
31f, 31g Hole
32 Second housing
32a Male thread portion
32b Hole
33 Third housing
33a Male thread portion
33b Hole
35 Drive unit
36 Transmission unit
39 Pipe
40, 40A Control unit
41, 41A Drive unit
41a Switching unit
42, 42A Contamination level measurement unit
43 Storage unit
44 Switching unit
45 Output unit
46 Display unit
50, 50A Control unit
51 Irradiation control unit
52 Measurement unit
53 Storage unit
54 Drive control unit
90 Rod-shaped member
91 Light-blocking slit

1. An adjustment method for adjusting a measurement device,
wherein the measurement device includes a light irradiation unit configured to irradiate a measurement flow path with light, and
a light receiving unit configured to continuously receive the light emitted from the light irradiation unit and having passed through the measurement flow path, and to obtain a continuous electric signal, the measurement device being configured to measure a contamination level of oil based on an electric signal obtained by the light receiving unit in a state in which the oil flows through the measurement flow path,
the adjustment method comprising
a first adjustment step of emitting light from the light irradiation unit in a state in which no oil flows through the measurement flow path, using a first pseudo signal for emitting light equivalent to light input to the light receiving unit when oil having a first contamination level as a given contamination level flows through the measurement flow path, and obtaining a measurement value for the first contamination level based on an electric signal obtained by receiving the emitted light by the light receiving unit.

2. The adjustment method according to claim 1, further comprising
a pseudo signal generating step of generating the first pseudo signal using a reference measurement device,
wherein the reference measurement device includes a reference light irradiation unit configured to continuously irradiate a reference measurement flow path with light, and a reference light receiving unit configured to continuously receive the light continuously emitted from the reference light irradiation unit and having passed through the measurement flow path, and to obtain a continuous electric signal,
the pseudo signal generating step includes
a first reference value measurement step of emitting light from the reference light irradiation unit in a state in which oil having the first contamination level flows through the reference measurement flow path, measuring an electric signal obtained by receiving the emitted light by the reference light receiving unit, and obtaining a first reference measurement value for the first contamination level, and
a first pseudo signal obtaining step of emitting light from the reference light irradiation unit, using a blinking signal for causing the reference light receiving unit to blink in a state in which no oil flows through the measurement flow path, and setting the blinking signal as the first pseudo signal when the electric signal obtained by the reference light receiving unit is equivalent to the electric signal having been obtained by the reference light receiving unit.

3. The adjustment method according to claim 2,
wherein the first reference value measurement step includes obtaining the first reference measurement value by smoothing an integrated value per unit time of the electric signal obtained by receiving light by the reference light receiving unit, and
the first pseudo signal obtaining step includes setting the blinking signal as the first pseudo signal when the integrated value per unit time of the electric signal obtained by receiving light by the reference light receiving unit is equivalent to the first reference measurement value.

4. The adjustment method according to claim 2,
wherein the first pseudo signal obtaining step includes, in a state in which no oil flows through the measurement flow path, adjusting an output level of the reference light irradiation unit in such a manner that a maximum value of an output value of a signal obtained by the reference light receiving unit is equivalent to a reference output value that is a maximum value of the electric signal obtained by receiving light by the reference light receiving unit in the first reference value measurement step, and after the adjusting of the output level, emitting light from the reference light irradiation unit and obtaining the first pseudo signal.

5. The adjustment method according to claim 4,
wherein the first adjustment step includes adjusting the output level of the light irradiation unit based on an adjustment result of the output level in the first pseudo signal obtaining step, and emitting light from the light irradiation unit, using the first pseudo signal at the output level having been adjusted.

6. The adjustment method according to claim 1,
wherein the measurement flow path includes a pipe at least partly made of glass, and
the state in which no oil flows through the measurement flow path is a state in which no liquid flows through the measurement flow path, a state in which a liquid other than oil flows through the measurement flow path, or a state in which a glass rod is provided instead of the pipe.

7. The adjustment method according to claim 6,
wherein the state in which a liquid other than oil flows through the measurement flow path is a state in which water, alcohol, or a mixture of water and alcohol flows through the measurement flow path.

8. An adjustment method for adjusting a measurement device including
a light irradiation unit configured to irradiate a measurement flow path with light, and
a light receiving unit configured to continuously receive the light emitted from the light irradiation unit and having passed through the measurement flow path, and to obtain a continuous electric signal,
the measurement device being configured to measure a contamination level of oil in a state in which the oil flows through the measurement flow path based on an electric signal obtained by the light receiving unit,
the adjustment method comprising
a first adjustment step of continuously emitting light from the light irradiation unit, while moving a rod-shaped member, provided with a plurality of light-blocking slits that do not transmit light, at a first speed within the measurement flow path in such a manner that the light is equivalent to light input to the light receiving unit when oil having a first contamination level as a given contamination level flows through the measurement flow path, and obtaining a measurement value for the first contamination level based on an electric signal obtained by receiving the light by the light receiving unit upon the emission.

9. The adjustment method according to claim 8, further comprising
a rod-shaped member adjustment step of adjusting a size and the number of the light-blocking slits and the first speed of the rod-shaped member, using a reference measurement device,
wherein the reference measurement device includes a reference light irradiation unit configured to continuously irradiate a reference measurement flow path with light, and a reference light receiving unit configured to continuously receive the light continuously emitted from the reference light irradiation unit and having passed through the measurement flow path, and to obtain a continuous electric signal,
the rod-shaped member adjustment step includes
a first reference value measurement step of emitting light from the reference light irradiation unit in a state in which oil having the first contamination level flows through the reference measurement flow path, measuring an electric signal obtained by receiving the emitted light by the reference light receiving unit, and obtaining a first reference measurement value for the first contamination level, and
an adjustment step of continuously turning on light from the reference light irradiation unit, obtaining an electric signal by the reference light receiving unit, while moving a provisional rod-shaped member, provided with a given size and number of the light-blocking slits, at a given provisional speed within the reference measurement flow path, and setting the provisional rod-shaped member and the provisional speed as the rod-shaped member and the first speed, respectively, when the electric signal is equivalent to the first reference measurement value.

10. The adjustment method according to claim 9,
wherein the first reference value measurement step includes obtaining the first reference measurement value by smoothing an integrated value per unit time of the electric signal obtained by receiving light by the reference light receiving unit, and
the adjustment step includes setting the provisional rod-shaped member and the provisional speed as the rod-shaped member and the first speed, respectively, when the integrated value per unit time of the electric signal obtained by receiving light by the reference light receiving unit is equivalent to the first reference measurement value.

11. An adjustment device that adjusts a measurement device including
a light irradiation unit configured to irradiate a measurement flow path with light, and a light receiving unit configured to continuously receive the light emitted from the light irradiation unit and having passed through the measurement flow path, and to obtain a continuous electric signal, the measurement device being configured to measure a contamination level of oil based on an electric signal obtained by the light receiving unit in a state in which the oil flows through the measurement flow path,
the adjustment device comprising:
an irradiation control unit configured to cause the light irradiation unit to emit light in a state in which no oil flows through the measurement flow path, using a first pseudo signal for emitting light equivalent to light input to the light receiving unit when oil having a first contamination level as a given contamination level flows through the measurement flow path; and
a measurement unit configured to obtain a measurement value for the first contamination level based on an electric signal obtained by receiving the light emitted from the light irradiation unit by the light receiving unit.

12. An adjustment device that adjusts a measurement device including
a light irradiation unit configured to irradiate a measurement flow path with light, and a light receiving unit configured to continuously receive the light emitted from the light irradiation unit and having passed through the measurement flow path, and to obtain a continuous electric signal, the measurement device being configured to measure a contamination level of oil based on an electric signal obtained by the light receiving unit in a state in which the oil flows through the measurement flow path,
the adjustment device comprising:
an irradiation control unit configured to cause the light irradiation unit to continuously emit light, while moving a rod-shaped member, provided with a plurality of light-blocking slits that do not transmit light, at a first speed within the measurement flow path in such a manner that the light is equivalent to light input to the light receiving unit when oil having a first contamination level as a given contamination level flows through the measurement flow path; and
a measurement unit configured to obtain a measurement value for the first contamination level based on an electric signal obtained by receiving the light emitted from the light irradiation unit by the light receiving unit.

13. A method for manufacturing a measurement device,
wherein the measurement device is configured to measure, in a state in which oil flows through a measurement flow path including a pipe, a contamination level of the oil based on an electric signal obtained by continuously receiving light, by a light receiving unit, emitted from a light irradiation unit configured to irradiate the measurement flow path with light, and having passed through the measurement flow path,
the method comprising steps of:
providing the light irradiation unit and the light receiving unit to sandwich the pipe;
causing the light irradiation unit to emit light in a state in which no oil flows through the measurement flow path, using a first pseudo signal for emitting light equivalent to light input to the light receiving unit when oil having a first contamination level as a given contamination level flows through the measurement flow path, and obtaining a measurement value for the first contamination level based on an electric signal obtained by receiving the emitted light by the light receiving unit; and
assembling a housing in which the measurement flow path, the light irradiation unit, and the light receiving unit are provided.

14. A method for manufacturing a measurement device,
wherein the measurement device is configured to measure, in a state in which oil flows through a measurement flow path including a pipe, a contamination level of oil based on an electric signal obtained by continuously receiving light, by a light receiving unit, emitted from a light irradiation unit configured to irradiate the measurement flow path with light, and having passed through the measurement flow path,
the method comprising the steps of:
providing the light irradiation unit and the light receiving unit to sandwich the pipe;
causing the light irradiation unit to continuously emit light in a state in which no oil flows through the measurement flow path, while moving a rod-shaped member, provided with a plurality of light-blocking slits that does not transmit light, at a first speed within the measurement flow path, in such a manner that the light is equivalent to light input to the light receiving unit when oil having a first contamination level as a given contamination level flows through the measurement flow path, and obtaining a measurement value for the first contamination level based on an electric signal obtained by receiving the light by the light receiving unit upon the emission; and
assembling a housing in which the measurement flow path, the light irradiation unit, and the light receiving unit are provided.

15. A measurement device comprising:
a light irradiation unit configured to irradiate a measurement flow path with light;
a light receiving unit configured to continuously receive the light emitted from the light irradiation unit and having passed through the measurement flow path, and to obtain a continuous electric signal;
a control unit configured to control lighting of the light irradiation unit, and obtain a measurement value based on a light reception result in the light receiving unit; and
a switching unit configured to switch between outputs from the light receiving unit in a case in which the light irradiation unit emits light in a state in which no oil flows through the measurement flow path, using a first pseudo signal for emitting light equivalent to light input to the light receiving unit when oil having a first contamination level as a given contamination level flows through the measurement flow path, and then a measurement value for the first contamination level is obtained based on an electric signal obtained by receiving the emitted light by the light receiving unit, and in a case in which a contamination level of oil is measured based on the measurement value for the first contamination level and an electric signal obtained by the light receiving unit in a state in which the oil flows through the measurement flow path.

16. A measurement device comprising:
a light irradiation unit configured to irradiate a measurement flow path with light;
a light receiving unit configured to continuously receive the light emitted from the light irradiation unit and having passed through the measurement flow path, and to obtain a continuous electric signal;
a control unit configured to control lighting of the light irradiation unit, and obtain a measurement value based on a light reception result in the light receiving unit; and
a switching unit configured to switch between outputs from the light receiving unit in a case in which the light irradiation unit continuously emits light, while moving a rod-shaped member, provided with a plurality of light-blocking slits that does not transmit light, at a first speed within the measurement flow path in such a manner that the light is equivalent to light input to the light receiving unit when oil having a first contamination level as a given contamination level flows through the measurement flow path, and then a measurement value for the first contamination level is obtained based on an electric signal obtained by receiving the light by the light receiving unit upon the emission, and a case in which a contamination level of oil is measured based on the measurement value for the first contamination level and an electric signal obtained by the light receiving unit in a state in which the oil flows through the measurement flow path.
